(19) United States
(12) Patent Application Publication
(10) Pub. No.: US 2020/0118684 A1
(43) Pub. Date: Apr. 16, 2020

(54) SYSTEMS AND METHODS FOR PREDICTIVE ORGAN TRANSPLANT SURVIVAL RATES

(71) Applicants: Georgia Tech Research Corporation, Atlanta, GA (US); Center for Disease Control, Atlanta, GA (US)

(72) Inventors: Ethan Joshua Mark, Atlanta, GA (US); David Goldsman, Atlanta, GA (US); Brian M. Gurbaxani, Atlanta, GA (US); Pinar Keskinocak, Atlanta, GA (US); Joel Sokol, Atlanta, GA (US)

(21) Appl. No.: 16/598,485

(22) Filed: Oct. 10, 2019

Related U.S. Application Data
(60) Provisional application No. 62/744,152, filed on Oct. 11, 2018.

(51) Int. Cl. G16H 50/20; G16H 50/70

(52) U.S. Cl. CPC G16H 50/20; G16H 50/70

(57) ABSTRACT

A method for predicting survival rates of a prospective organ recipient is disclosed. The method may include receiving a first dataset that includes characteristics of previous persons in need of a transplant that received or did not receive the organ transplant and their respective actual survival rates. The method may further include receiving a second dataset that includes characteristics of a prospective organ recipient and characteristics of a donor organ available for an organ transplant into the prospective organ recipient. The method may then calculate the estimated survival rates over a predetermined of the prospective organ recipient based on whether the prospective organ recipient has the organ transplanted. Next, a graph may be generated showing the estimated survival rates of the prospective organ recipient based on whether the prospective organ recipient receives and does not receive the organ transplant, which may be displayed a graphical user interface.

### CROSS-REFERENCE TO RELATED APPLICATION

This application claims the benefit of, and priority under 35 U.S.C. § 119(e) to, U.S. Provisional Patent Application No. 62/744,152, entitled “Infectious Risk Donor (IRD) Organ Decision Support Tool and Survival Curve Estimation,” filed Oct. 11, 2018, the contents of which are hereby incorporated by reference herein in their entirety as if fully set forth below.

### FIELD OF THE INVENTION

The presently disclosed subject matter relates generally to systems and methods for predictive organ transplant survival rates and, more particularly, to systems and methods for determining an organ transplant recipient and/or predicting organ transplant survival rates for individual prospective organ transplant recipients.

### BACKGROUND

Presently, over 100,000 people in the United States are on the waiting list for an organ transplant. Clearly, the demand for organs greatly exceeds the available number of organs available. As a result, over 20 people per day die in the United States while waiting on an organ transplant. In instances where organs are received from a recently deceased donor, there are usually only hours before the organs must be transplanted. Because of this, recipients must be determined quickly, and those recipients must quickly accept a donor organ with little time to analyze several considerations. Some considerations that a prospective recipient might want to consider include: whether the organ is an infectious risk donor (IRD) organ (e.g., donor died of an overdose, donor had hepatitis C, etc.); chances of surviving the procedure; life expectancy with the organ transplant; and/or whether foregoing the organ in favor of another organ would be more beneficial.

During extremely time-sensitive moments, customized survival rates may assist prospective recipients in their decision to accept or deny the organ. Also, the customized survival rates may help medical personnel in more efficiently selecting and contacting prospective recipients on the organ transplant waitlist. Further, by better informing prospective organ recipients of the survival rates of a specific organ, IRD organs that are often discarded, may be used, which may provide a dual benefit; a reduction to the organ transplant waitlist and a healthy organ for the prospective recipient.

Accordingly, there is a need for an improved system and method for providing prospective recipient specific estimated survival rates of an organ transplant.

### SUMMARY

Aspects of the disclosed technology include systems and methods for predicting survival rates of a prospective organ recipient. Consistent with the disclosed embodiments, the methods may include one or more processors, transceivers, computing devices, user devices, or databases. One exemplary method may include calculating multiple estimated survival rates (e.g., a first and second set of estimated survival rates) for the prospective organ recipient. The method may include the computing device receiving a first dataset that includes characteristics of previous prospective organ transplant recipients, characteristics of previous persons in need of an organ transplant that did not receive the organ transplant, a first set of actual survival rates of the previous prospective organ transplant recipients, and/or a second set of actual survival rates of the previous persons in need of an organ transplant that did not receive the organ transplant.

The method may further include the computing device receiving a second dataset that includes characteristics of the prospective organ recipient, characteristics of a first organ from a first organ donor available for an organ transplant into the prospective organ recipient, and/or an estimated wait time for a second organ from a second organ donor available for transplant into the prospective organ recipient to become available.

Based on at least a portion of the first dataset and at least a portion of the second dataset, the computing device may calculate the first set of estimated survival rates of the prospective organ recipient over predetermined time periods. The first set of estimated survival rates may be based on the organ recipient foregoing the organ transplant with the first organ. Further, based on at least a portion of the first dataset and at least a portion of the second dataset, the method may include calculating a second set of estimated survival rates of the prospective organ recipient over the predetermined time periods. Here, the second set of estimated survival rates may be based on the organ recipient receiving the organ transplant with the first organ. Once calculated, the method may include generating a first graph of the first and second set of estimated survival rates of the prospective organ recipient over the predetermined periods. The method may also include displaying the first graph as a graphical user interface on the computing device.

In some embodiments, the characteristics of the previous prospective organ transplant recipients, the characteristics of previous organ donors received by the previous prospect organ transplant recipients, the characteristics of the prospective organ recipient, the characteristics of previous persons in need of an organ transplant that did not receive the organ transplant, and the characteristics of the first organ from the first organ donor available for an organ transplant may include age, sex, blood type, transplant region, height, weight, a current medical state, a medical condition (e.g., high cholesterol, diabetes), or an organ type (e.g., kidney, liver, lungs, or a heart).

According to some embodiments, the organ type may further include a status of the organ type (e.g., infection-risk disease (IRD) or non-IRD).

In some embodiments, the computing device may send a message to a user device associated with the prospective organ transplant recipient where the message includes an offer and/or details about the first organ. The computing device may receive from the user device associated with the prospective organ transplant recipient, a response that includes an acceptance or a denial of the offer of the first organ.

In some embodiments, the message may include instructions that cause a graphical user interface of the user device to display the graph.

In some embodiments, the computing device may send the first graph to the user device as part of the message or separately.

In some embodiments, calculating the first set of estimated survival rates may be based in part on the organ type. For example, when the organ type is a kidney, calculating the first set of estimated survival rates may comprise assigning a respective weight to each of the characteristics of previous persons in need of an organ transplant that did not receive the organ transplant, comparing the characteristics of the prospective organ recipient to each of the characteristics of previous persons in need of an organ transplant that did not receive the organ transplant to determine at least partial congruence, calculating an average set of survival rates for a set of congruent previous persons based on the second set of actual survival rates for each of the congruent previous persons, and setting the average set of survival rates as the first set of estimated survival rates.

In some embodiments, when the organ type is a liver, heart, and/or lungs, calculating the first set of estimated survival rates may comprise assigning a respective weight to each of the characteristics of previous persons in need of an organ transplant that did not receive the organ transplant, comparing the characteristics of the prospective organ recipient to each of the characteristics of the previous persons in need of an organ transplant that did not receive the organ transplant to determine at least partial congruence, calculating a first transplant score for each of the prospective organ recipients by totaling the respective weights of congruent characteristics, determining an average set of survival rates for the previous persons in need of an organ transplant that did not receive the organ transplant, calculating new shifted survival rates by multiplying the average set of survival rates by the transplant score for each prospective organ recipient, and setting the new shifted survival rates as the first set of estimated survival rates.

In some embodiments, rather than determining a set of previous persons, the method may include identifying a most congruent previous person—i.e., the congruent previous person in need of an organ transplant that did not receive the organ transplant having the most congruent characteristics to the prospective organ recipient, identifying the second set of actual survival rates for the most congruent previous person, and setting the second set of actual survival rates for the most congruent previous person as the first set of estimated survival rates.

In some embodiments, calculating the second set of estimated survival rates may be based in part on the organ type. For example, when the organ type is a kidney, calculating the second set of estimated survival rates may comprise assigning a respective weight to each of the characteristics of the previous organ transplant recipients, comparing the characteristics of the prospective organ recipient to each of the characteristics of previous organ transplant recipients to determine at least partial congruence, calculating an average set of survival rates for a set of congruent previous persons based on the actual survival rates for each of the congruent previous persons, and setting the average set of actual survival rates as the second set of estimated survival rates.

In some embodiments, when the organ type is a liver, heart, and/or lungs, calculating the second set of estimated survival rates may comprise assigning a respective weight to each of the characteristics of the previous organ transplant recipients, comparing the characteristics of the prospective organ recipient to each of the characteristics of the previous organ transplant recipients to determine at least partial congruence, calculating a transplant score for each of the prospective organ recipients by totaling the respective weights of congruent characteristics, determining an average set of survival rates for the previous organ transplant recipients, calculating new shifted survival rates by multiplying the average set of survival rates by the transplant score for each prospective organ recipient, and setting the new shifted survival rates as the second set of estimated survival rates.

According to some embodiments, upon receiving new data of prospective recipients receiving or foregoing organ transplants, the computing device may update the predictive algorithm by reassigning the respective weights assigned to at least one of: (i) one or more of the characteristics of previous persons in need of an organ transplant that did not receive the organ transplant; or (ii) one or more characteristics of previous prospective organ transplant recipients.

According to some embodiments, the computing device may send, to a server, the graph for retrieval by an application of a user device.

Another exemplary method may include determining an organ recipient for an organ transplant. The method may include the computing device receiving a first dataset that includes characteristics of previous prospective organ transplant recipients, characteristics of previous organs received by the previous prospective organ transplant recipients, characteristics of previous persons in need of an organ transplant that did not receive the organ transplant, a first set of actual survival rates of the previous prospective organ transplant recipients, and/or a second set of actual survival rates of the previous persons in need of an organ transplant that did not receive the organ transplant.

The computing device may also receive a second dataset that includes characteristics of each of a plurality of prospective organ recipients and characteristics of an organ from an organ donor available for an organ transplant into at least one of the plurality of prospective organ recipients.

The method may further include the computing device executing a predictive algorithm to calculate a first set of estimated survival rates for each of the plurality of prospective organ recipients over a predetermined period based at least on a portion of the first dataset and at least a portion of the second dataset. The first set of estimated survival rates may be based on each of the plurality of prospective organ recipients foregoing the organ transplant. Also, the method may include the computing device executing the predictive algorithm to calculate a second set of estimated survival rates for each of the plurality of prospective organ recipients over the predetermined period based at least on a portion of the first dataset and at least a portion of the second data. The second set of estimated survival rates may be based on each of the plurality of prospective organ recipients receiving the organ transplant. Next, the method may identify an organ recipient from amongst the plurality of prospective organ recipients. The organ recipient may be one of the plurality of prospective organ recipients having the highest second set of estimated survival rates over the predetermined period. The computing device may generate a first graph of the first set of estimated survival rates of the organ recipient and the second set of estimated survival rates for the organ recipient over the predetermined period, which may be sent to a first user device associated with the organ recipient along with an offer of an organ. In turn, the computing device may receive a first response from the first user device that includes an acceptance or a denial of the offer of the organ.

In some embodiments, the computing device may send, to a server, the first graph for retrieval by an application of the first user device associated with the organ recipient.

According to some embodiments, the characteristics of the previous prospective organ transplant recipients, the characteristics of previous organ donors received by the previous prospect organ transplant recipients, the characteristics of the prospective organ recipient, the characteristics of previous persons in need of an organ transplant that did not receive the organ transplant, and/or the characteristics of the organ from the organ donor available for an organ transplant may include age, sex, blood type, height, weight, transplant region, a medical condition, and/or or an organ type. The organ type may include a kidney, liver, lungs, and/or a heart.

In some embodiments, the organ type may further include a status of the organ type, where the status is either infection-risk disease (IRD) or non-IRD.

In some embodiments, after receiving a denial from the first user device, the computing device may determine a next organ recipient from the plurality of prospective organ recipients. The computing device may also send, to a second user device associated with the next organ recipient, a second message that includes an offer of the organ. In turn, the computing device may receive a second response that includes an acceptance or a denial from the second user device.

According to some embodiments, the method may include the computing device generating a second graph of the first set of estimated survival rates of the next organ recipient and the second set of estimated survival rates for the next organ recipient over the predetermined period, which may be sent to the second user device along with the second message including the offer of the organ. Next, the computing device may receive the second response from the second user device that includes an acceptance or a denial of the offer of the organ.

In some embodiments, the computing device may send, to a server, the second graph for retrieval by an application of the second user device.

Further features of the disclosed design, and the advantages offered thereby, are explained in greater detail hereinafter with reference to specific embodiments illustrated in the accompanying drawings, wherein like elements are indicated be like reference designators.

### BRIEF DESCRIPTION OF THE DRAWINGS

Reference will now be made to the accompanying drawings, which are not necessarily drawn to scale, are incorporated into and constitute a portion of this disclosure, illustrate various implementations and aspects of the disclosed technology, and, together with the description, serve to explain the principles of the disclosed technology. In the drawings:

FIG. 1 is a diagram of an example system for determining predictive organ transplant survival rates, in accordance with some examples of the present disclosure;

FIG. 2 is an example flow chart of a method for determining predictive organ transplant survival rates, in accordance with some examples of the present disclosure;

FIGS. 3A-B are example flow charts of a method for identifying an organ recipient for an organ transplant from a plurality of prospective organ recipient, in accordance with some examples of the present disclosure;

FIG. 4 is a component diagram of an example of a user device, in accordance with some examples of the present disclosure;

FIG. 5 is a component diagram of an example of a computing device in accordance with some examples of the present disclosure; and

FIG. 6 is a screen of a device with a graphical user interface, in accordance with some examples of the present disclosure.

### DETAILED DESCRIPTION

Some implementations of the disclosed technology will be described more fully with reference to the accompanying drawings. This disclosed technology can be embodied in many different forms, however, and should not be construed as limited to the implementations set forth herein. The components described hereinafter as making up various elements of the disclosed technology are intended to be illustrative and not restrictive. Many suitable components that would perform the same or similar functions as components described herein are intended to be embraced within the scope of the disclosed electronic devices and methods. Such other components not described herein can include, but are not limited to, for example, components developed after development of the disclosed technology.

It is also to be understood that the mention of one or more method steps does not imply that the methods steps must be performed in a particular order or preclude the presence of additional method steps or intervening method steps between the steps expressly identified.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

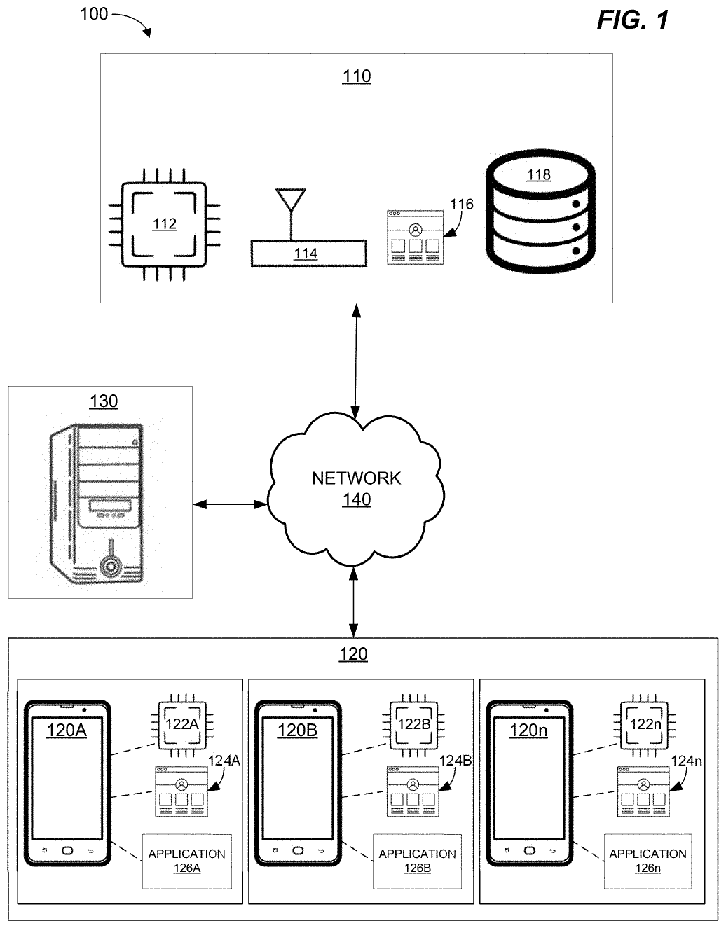

FIG. 1 shows an example system 100 that may implement certain methods for predictive survival rates of prospective organ transplant recipients. As shown in FIG. 1, in some implementations the system 100 may include one or more user devices 120A-120n, which may include one or more processors 122, a graphical user interface (GUI) 124, an application 126, among other things. The system 100 may further include a computing device 110, which may include one or more processors 112, a transceiver 114, a GUI 116, and a database 118, among other things. The computing device 110 may belong to a hospital, medical provider, an organ donor registry, or another institution involved in, for example, selecting and/or notifying prospective organ transplant recipients of a matching organ. The system 100 may also include an external server 130, which may belong to the donor registry, for example, or may belong to another third-party. Further, the system 100 may include the network 140 that may include a network of interconnected computing devices such as, for example, an intranet, a cellular network, or the Internet.

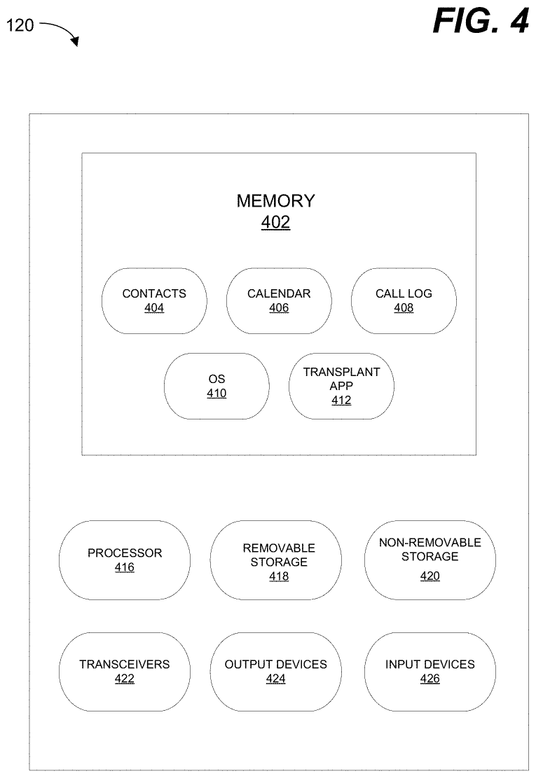

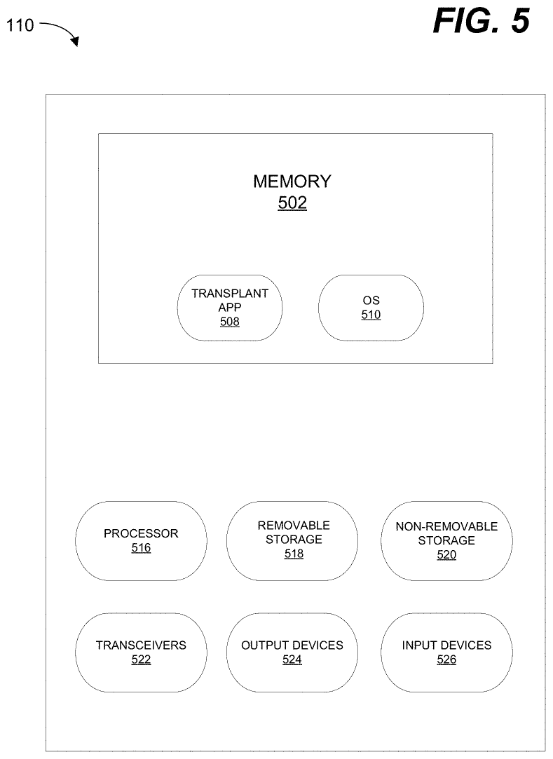

The user device 120 may be, for example, a personal computer, a smartphone, a laptop computer, a tablet, or other computing device. An example computer architecture that may be used to implement the user device 120 is described below with reference to FIG. 4. The computing device 110 may include one or more physical or logical devices (e.g., servers) or drives and may be implemented as a single server, a bank of servers (e.g., in a “cloud”), run on a local machine, or run on a remote server. An example computer architecture that may be used to implement the computing device 110 is described below with reference to FIG. 5.

The computing device 110 may calculate estimated survival rates over predetermined periods for the prospective organ recipient based on whether the prospective organ recipient accepts the first donor organ (e.g., first set of estimated survival rates) or denies the first donor organ while waiting for a second donor organ (e.g., second set of estimated survival rates). To accomplish this, the computing device 110 may receive a first dataset that includes characteristics of previous prospective organ transplant recipients, characteristics of previous organs received by the previous prospective organ transplant recipients, characteristics of previous persons in need of an organ transplant that did not receive the organ transplant, a first set of survival rates of the previous prospective organ transplant recipients, and/or a second set of survival rates of the previous persons in need of an organ transplant that did not receive the organ transplant.

Further, the computing device 110 may receive a second dataset that includes characteristics of the prospective organ recipient, characteristics of a first organ from a first organ donor available for an organ transplant into the prospective organ recipient, and an estimated wait time for a second organ from a second organ donor available for transplant into the prospective organ recipient to become available. The first dataset and/or the second dataset may be stored within the database 118 or may be received from the external server 130 (e.g., a national registry of prospective organ recipients and/or one or more hospitals receiving donor organs).

The characteristics of the previous prospective organ transplant recipients, the characteristics of previous organ donors received by the previous prospect organ transplant recipients, the characteristics of the prospective organ recipient, the characteristics of previous persons in need of an organ transplant that did not receive the organ transplant, and the characteristics of the first organ from the first organ donor available for an organ transplant may include age, gender, blood type, transplant region, height, weight, a medical condition (e.g., diabetic), medical history, family medical history, ethnicity, race, and/or the like. Similarly, the characteristics of the previous organs received by the previous prospective organ transplant recipients may include certain characteristics of a donor of the organ including the donor's age, gender, blood type, height, weight, a medical condition (e.g., sickle cell disease), medical history, family medical history, ethnicity, race, current medical condition, and/or the like. Further, the characteristics of the previous organs received by the previous prospective organ transplant recipients may include an organ type (e.g., a heart, kidneys, lungs, liver, etc.). The organ type may further include a status of whether the organ is infection-risk disease (IRD) or non-IRD. An organ may be classified as IRD when the donor recently injected drugs, was incarcerated, had sexual intercourse for drugs or money, or of course had an infectious disease such as hepatitis C or HIV.

To calculate the first set of estimated survival rates, the computing device 110 may identify one or more previous persons in need of an organ transplant that did not receive the organ transplant from the first dataset that has similar attributes to the prospective organ transplant recipient. This may include the computing device 110 comparing the characteristics of the prospective organ transplant recipient and the characteristics of previous persons in need of an organ transplant that did not receive the organ transplant for at least partial congruence. For instance, a 55-year-old woman, who is 5′2″, weighs 125 lbs, and needs a kidney may be congruent with previous women over 50 years of age, between 5′0″ and 5′5″, weighing between 110 and 140 lbs, and who were in need of kidney, but did not receive the donor organ. Of course, in determining congruence, each of the characteristics may be weighted differently or each may have the same value.

Next, the computing device 110 may identify, from the first dataset, the second set of actual survival rates of the congruent women that did not receive the organ transplant. The actual survival rates of these women who survived without the donor organ may be an average, and/or may be categorized by a predetermined time period, for example, the computing device 110 may determine that 80% of these women survived at least two years without the donor organ, that only 50% of these women survived four years without the donor organ, that only 20% of these women survived over six years without the donor organ, and that no woman lived over ten years without the donor organ. Using this information, the computing device 110 may also analyze data from the second dataset such as an estimated wait time for a second donor organ to become available for transplant. Therefore, if the prospective organ donor recipient decides to forego the organ transplant, for example, because the organ is an IRD organ, the computing device 110 may determine the likelihood (e.g., 30% chance of survival) of the prospective organ donor recipient surviving over the estimated wait time for the second organ. Further, the computing device 110 may determine the first set of estimated survival rates—i.e., the likelihood of the prospective organ donor recipient surviving over other periods (e.g., one year, two years, five years, ten years, etc.).

The second set of estimated survival rates may be based on the prospective organ recipient receiving the donor organ. The computing device 110 may calculate the second set of estimated survival rates based on the first dataset and/or the second dataset. Similar to above, this may include the computing device 110 identifying, from the first dataset, characteristics of previous prospective organ transplant recipients, and from the second dataset, characteristics of the prospective organ recipient. The computing device 110 may compare the characteristics of previous prospective organ transplant recipients to the characteristics of the prospective organ recipient to determine at least partial congruence. Next, the computing device 110 may identify the actual survival rates of the congruent previous prospective organ transplant recipients from the first set of actual survival rates to determine the second set of estimated survival rates.

The computing device 110 may also generate a graph that includes the first set of estimated survival rates and the second set of estimated survival rates over the predetermined periods, which may be displayed by the GUI 116. The computing device 110 may send the graph to the external server 130, which may provide the graph for download to a user device (e.g., user device 120). The computing device 110 may also send instructions to the user device 120 that cause the user device 120 to display the graph as the GUI 124. The instructions may be sent separately or as part of a message sent to the user device 120. The message may include details about the donor organ and/or an option to approve or deny an offer of the donor organ.

The user device 120 may send a response to the message to the computing device 110 that includes an approval or denial of the offer for the donor organ. The prospective organ recipient may also input other characteristics about herself at the GUI 124, which may also be sent to the computing device 110 to be included with the second dataset.

The computing device 110 may continually refine the predictive algorithm based at least in part on new data of prospective recipients receiving or foregoing organ transplants, such that the predictive algorithm may improve at identifying a donor organ for a prospective organ recipient. For example, upon receiving new data of prospective recipients receiving or foregoing organ transplants, the computing device 110 may update the predictive algorithm by reassigning the respective weights assigned to one or more of the characteristics of previous persons in need of an organ transplant that did not receive the organ transplant and/or one or more characteristics of previous prospective organ transplant recipients.

Determining an organ recipient, for a donor organ, may also be performed by the computing device 110. To do so, the computing device 110 may receive a first dataset that includes characteristics of previous prospective organ transplant recipients, characteristics of previous organs received by the previous prospective organ transplant recipients, characteristics of previous persons in need of an organ transplant that did not receive the organ transplant, a first set of survival rates of the previous prospective organ transplant recipients, and/or a second set of survival rates of the previous persons in need of an organ transplant that did not receive the organ transplant. Of course, the first dataset may be retrieved from the database 118 or received from an external source (e.g., the external server 130).

Next, the computing device 110 may receive a second dataset that includes characteristics of each of a plurality of prospective organ recipients and characteristics of an organ from an organ donor available for an organ transplant into at least one of the plurality of prospective organ recipients. Similar to the first dataset, the second dataset may be retrieved from the database 118 or received from an external source (e.g., the external server 130).

The computing device 110 may calculate a first set of estimated survival rates (i.e., the estimated survival rates while foregoing the organ transplant) for each of the prospective organ recipients based on at least on a portion of the first dataset and at least a portion of the second dataset. More specifically, the computing device 110 may compare the characteristics of each of the prospective organ recipients to the characteristics of the previous persons in need of an organ transplant that did not receive the organ transplant for at least partial congruence. The computing device 110 may identify the previous persons most congruent to each of the prospective organ recipients and include them in a respective set of congruent previous persons. Next, the computing device 110 may identify the second set of actual survival rates for each previous person in the respective set of congruent previous persons to calculate an average of the actual set of second survival rates over the predetermined period, which the computing the device 110 may set as the first set of estimated survival rates.

Further, the computing device 110 may calculate a second set of estimated survival rates (i.e., the estimated survival rates if the organ transplant is received) for each of the prospective organ recipients based on at least on a portion of the first dataset and at least a portion of the second dataset. This may involve the computing device 110 comparing the characteristics of each of the prospective organ recipients to the characteristics of the previous prospective organ transplant recipients to determine at least partial congruence. Additionally, the computing device 110 may identify a set of congruent previous prospective organ transplant recipients for each prospective organ recipient, and then identify the first set of actual survival rates for each of the congruent previous prospective organ transplant recipients. An average of the first set of actual survival rates may then be calculated for each of the congruent previous prospective organ transplant recipients, which the computing device 110 may set as the second set of estimated survival rates for each respective prospective organ recipient.

The computing device 110 may then analyze the second set of estimated survival rates for each prospective organ recipient to determine the prospective organ recipient most likely to live the longest with the transplant—i.e., the prospective organ recipient with the highest second set of estimated survival rates over the predetermined period. Then, the computing device 110 may generate a first graph that includes the first set of estimated survival rates of the organ recipient and the second set of estimated survival rates for the organ recipient over the predetermined period. The first graph and a first message including an offer of the organ and/or details about the organ (e.g., characteristics of the organ from the organ donor available for an organ transplant) may be sent by the computing device 110 to a first user device (e.g., user device 120A). In turn, the computing device 110 may receive a first response to the first message from the first user device 120A, which may be an acceptance or a denial of the offer. The computing device 110 may also send the first graph to the external server 130 for retrieval by an application of the first user device 120A.

The computing device 110 may update the predictive algorithm, which may refine the predictive algorithm such that it can better identify prospective organ recipients likely to accept the organ. As previously mentioned, updating the predictive algorithm many include assigning and/or reassigning weights of the one or more of the characteristics of previous persons in need of an organ transplant that did not receive the organ transplant and/or one or more characteristics of previous prospective organ transplant recipients.

When the organ recipient rejects the offer for the organ, the computing device 110 may then determine a next organ recipient from the prospective organ recipients, which may be the prospective organ recipient with the second highest second set of estimated survival rates over the predetermined period. The computing device 110 may identify the first set of estimated survival rates for the next organ recipient from the first set of estimated survival rates, and the second set of estimated survival rates for the next organ recipient from the second set of estimated survival rates, which may be included in a second graph generated by the computing device 110.

The computing device 110 may send the second graph and/or a second message to a second user device (e.g. user device 120B) associated with the next organ recipient that includes an offer of the organ and/or details about the organ. The computing device 110 may also send the second graph to the external server 130 for retrieval by application 126B of the second user device 120B. Of course, the next organ recipient may view the second graph to help decide whether to accept the organ. The next organ recipient may send, from the second user device 120B, a second response to the offer of the organ that either accepts or denies the offer.

The external server 130 may store personally identifiable data for each of the prospective organ recipients (e.g., biometric data (retinal data, fingerprint data, facial recognition data), username and password, an established gesture). Also, the external server 130 may provide, for download, various graphs (e.g., the first graph) and information (e.g., characteristics of the organ from the organ donor available for an organ transplant), which may require the organ recipient to enter matching personally identifiable data before allowing the download.

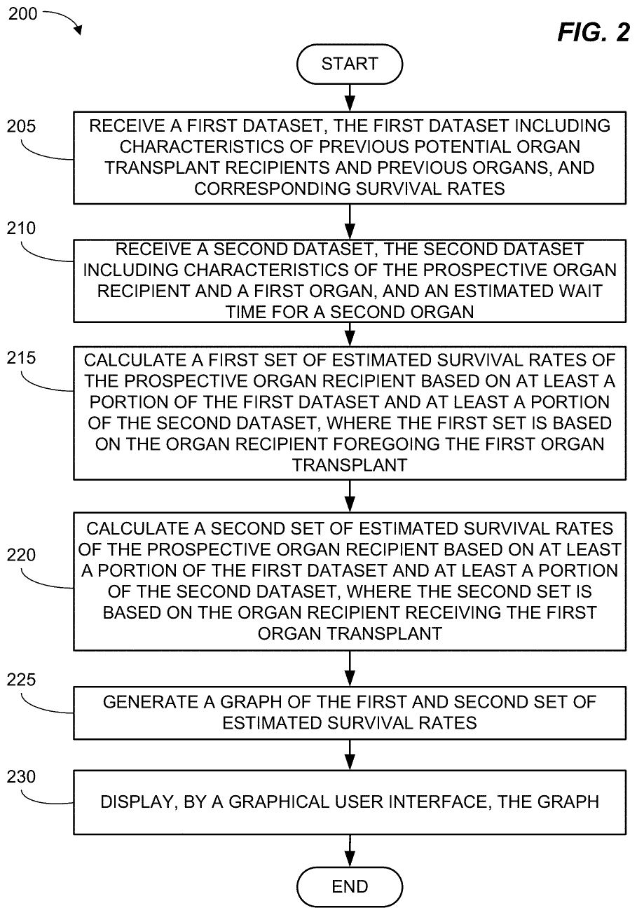

FIG. 2 shows an example flow chart of a method for determining predictive organ transplant survival rates. The method 200 is written from the perspective of the computing device 110, which may communicate with the user device 120 and/or the external server 130. The computing device 110 may predict transplant survival rates, generate the transplant survival rates as a graph, and transmit the graph either directly to the prospective organ recipient (e.g., user device 120), or indirectly (e.g., to the external server 130), where the prospective organ recipient may obtain the graph.

At 205, the computing device 110 may receive a first dataset from the database 118, the external server 130, and/or the user device 120. It should be noted that the first dataset may be received piecemeal from the aforementioned sources and later combined at the computing device 110. For example, each of the previous prospective organ recipients and/or each of the previous persons in need of an organ transplant that did not receive the organ transplant may have sent their respective characteristics from an associated user device to the computing device 110. The first dataset may include characteristics of previous prospective organ transplant recipients, characteristics of previous organs received by the previous prospective organ transplant recipients, characteristics of previous persons in need of an organ transplant that did not receive the organ transplant, a first set of actual survival rates of the previous prospective organ transplant recipients, and/or a second set of actual survival rates of the previous persons in need of an organ transplant that did not receive the organ transplant.

At 210, the computing device 110 may receive a second dataset, which also may be received from the database 118, the external server 130, and/or the user device 120. The second dataset may include characteristics of the prospective organ recipient, characteristics of a first organ from a first organ donor available for an organ transplant into the prospective organ recipient, and/or an estimated wait time for a second organ from a second organ donor available for transplant into the prospective organ recipient to become available.

At 215, the computing device 110 may calculate a first set of estimated survival rates of the prospective organ recipient over predetermined time periods. The first set of estimated survival rates of the prospective organ recipient may be based on the prospective organ recipient foregoing the organ transplant. For example, the computing device 110 may predict the prospective organ recipient's chances of survival without the organ transplant over a period of days, months, and/or years. The computing device 110 may accomplish this by comparing the characteristics of the prospective organ recipient to the characteristics of the previous persons in need of an organ transplant that did not receive the organ transplant to identify previous person(s) in need of an organ transplant that did not receive the organ transplant most similar to the prospective organ recipient (e.g., the previous persons having the most congruent characteristics of the prospective organ). Further, the computing device 110 may identify the second set of actual survival rates of the previous persons. The first set of estimated survival rates may be the second set of actual survival rates of the most congruent previous person, or the first set of estimated survival rates may be an average of the second set of actual survival rates of a group of previous persons most congruent to the prospective organ recipient.

At 220, the computing device 110 may calculate a second set of estimated survival rates of the prospective organ recipient over predetermined time periods, which may be based on the prospective organ recipient receiving the organ transplant. To predict the second set of estimated survival rates of the prospective organ recipient over the predetermined periods, the computing device 110 may compare the characteristics of the prospective organ recipient to the characteristics of the previous prospective organ transplant recipients to identify previous prospective organ transplant recipient(s) most similar to the prospective organ recipient. Further, the computing device 110 may identify the first set of actual survival rates of the most congruent previous prospective organ recipient or each of a group of previous organ recipients most congruent to the prospective organ recipient. In the former example, the computing device 110 may assign the first set of actual survival rates of the most closely congruent previous prospective organ recipient as the second set of estimated survival rates of the prospective organ recipient. In the latter example, the computing device 110 may determine an average first set of actual survival rates for the group and then assign that as the second set of estimated survival rates of the prospective organ recipient.

At 225, the computing device 110 may generate a graph of the first and a second set of estimated survival rates of the prospective organ recipient over predetermined time periods, which may be displayed as the GUI 116, at 230. In some embodiments, the computing device 110 may send the graph, or instructions that cause the graph to be displayed, to the external server 130, which may a user device (e.g., user device 120) to download the graph. Once the prospective organ recipient is able to view the graph, she may be better informed on whether to accept the organ.

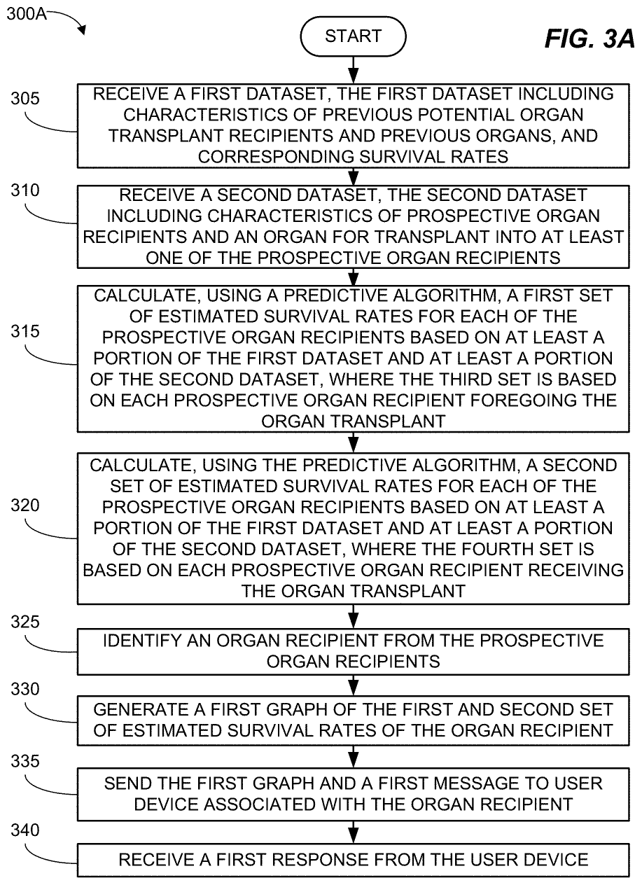

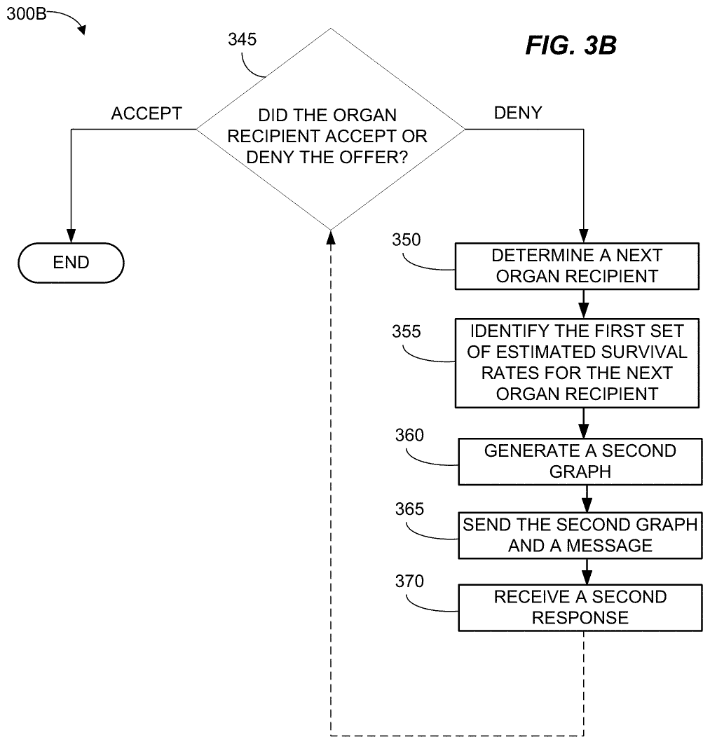

FIGS. 3A-B are example flow charts of a method for identifying an organ recipient for an organ transplant from a plurality of prospective organ recipients. The method 300A and 300B may be performed by the computing device 110 in communication with the first user device 120A, the second user device 120B, and the external server 130. The computing device 110 may determine a prospective organ recipient who will have the longest life expectancy if the organ is transplanted. Further, the computing device 110 may offer the organ to the prospective organ recipient, and if the prospective organ recipient denies the offer, the computing device 110 may continue to identify prospective organ recipients until the organ is accepted for a transplant.

At 305, the computing device 110 may receive a first dataset from the database 118, the external server 130, and/or the user device 120. As with the first dataset mentioned in FIG. 2, the first dataset may include characteristics of previous prospective organ transplant recipients, characteristics of previous organs received by the previous prospective organ transplant recipients, characteristics of previous persons in need of an organ transplant that did not receive the organ transplant, a first set of actual survival rates of the previous prospective organ transplant recipients, and/or a second set of actual survival rates of the previous persons in need of an organ transplant that did not receive the organ transplant.

At 310, the computing device 110 may receive a second dataset, that like the first dataset, may be received from the database 118, the external server, and/or the user device 120. The second dataset may include characteristics of each of a plurality of prospective organ recipients and/or characteristics of an organ from an organ donor available for an organ transplant into at least one of the plurality of prospective organ recipients.

At 315, the computing device executing the predictive algorithm may calculate a first set of estimated survival rates (e.g., likelihood of survival of the predetermined periods while foregoing the organ transplant) for each of the plurality of prospective organ recipients over a predetermined period (e.g., one year, five years, ten years, and twenty years) based at least on a portion of the first dataset and at least a portion of the second dataset. More specifically, the computing device 110 may compare characteristics of each of the plurality of prospective organ recipients to the characteristics of the previous persons in need of an organ transplant that did not receive the organ transplant to determine at least partial congruence, i.e., previous people in need of an organ transplant similar to the prospective organ recipient. Next, for each of the prospective organ recipients, the computing device 110 may identify the second set of actual survival rates of the congruent previous person(s) to determine either an average of the second set of actual survival rates, or the second set of actual survival rates of the most congruent previous person; either of which may be set as the first set of estimated survival rates.

At 320, the computing device 110 may calculate the second set of estimated survival rates (e.g., likelihood of survival of the predetermined periods if the organ transplant is performed) for each of the plurality of prospective organ recipients over the predetermined period. To do so, the computing device 110 may compare characteristics of each of the plurality of prospective organ recipients to the characteristics of previous prospective organ transplant recipients to determine at least partial congruence. Further, the computing device 110 may compare the characteristics of the previous organs received by the congruent previous prospective organ transplant recipients to the characteristics of the organ from the organ donor available for the organ transplant to determine at least partial congruence. Next, the computing device 110 may identify the previous prospective organ transplant recipients congruent to both of the previous criteria, and then identify the first set of actual survival rates of the congruent previous prospective organ transplant recipient(s). Then, for each of the prospective organ recipients, the computing device 110 may set the second set of estimated survival rates as the first set of actual survival rates of the most congruent previous organ transplant recipient, or as an average of the first set of actual survival rates of a group most congruent to the prospective organ recipient (e.g., five previous organ recipients who have the most characteristics congruent to the prospective organ recipient).

At 325, the computing device 110 may identify the prospective organ recipient having the highest second set of estimated survival rates of the predetermined period (i.e., the prospective organ recipient most likely to live the longest with the organ transplant) as the organ recipient. The computing device 110 may then generate a first graph of the first set of estimated survival rates of the organ recipient and the second set of estimated survival rates of the organ recipient over the predetermined period at 330. At 335, the computing device 110 may send the first graph and a first message that includes details about the organ and an offer of the organ to the first user device 120A. In response, at 340, the computing device 110 may receive a first response from the first user device 120A that includes an acceptance or a denial of the offer of the organ.

At 345, the computing device 110 may determine whether the first response is an acceptance or a denial of the organ. When the organ recipient sends the first response that denies the organ, the computing device 110, at 350, may then determine a next organ recipient. To do so, the computing device 110 may identify the prospective organ recipient having the second highest second set of estimated survival rates, and set that prospective organ recipient as the next organ recipient. At 355, the computing device 110 may identify the first set of estimated survival rates for the next organ recipient, which may be included along with the first set of estimated survival rates in a generated second graph, at 360. At 365, the computing device 110 may then send the second graph, and a second message that includes details about the organ and an offer of the organ, to a second user device 120B associated with the next organ recipient. At 370, the computing device 110 may receive a second response to the second message from the second user device 120B. Of course, the second response may be an acceptance or a denial of the organ, which may be determined at 345 with the method repeating thereof when the response is a denial.

As shown in FIG. 4, some, or all, of the system 100 and methods 200, 300A and 300B may be performed by, and/or in conjunction with, the user device 120. In some examples, the user device 120 may comprise, for example, a cell phone, a smart phone, a tablet computer, a laptop computer, a desktop computer, a sever, or other electronic device. The user device 120 may be a single server, for example, or may be configured as a distributed, or “cloud,” computer system including multiple servers or computers that interoperate to perform one or more of the processes and functionalities associated with the disclosed examples. One of skill in the art will recognize, however, that the system 100 and methods 200, 300A and 300B may also be used with a variety of other electronic devices, such as, for example, tablet computers, laptops, desktops, and other network (e.g., cellular or internet protocol (IP) network) connected devices from which a call may be placed, a text may be sent, and/or data may be received. These devices are referred to collectively herein as the user device 120. The user device 120 may comprise a number of components to execute the above-mentioned functions and apps. As discussed below, the user device 120 comprise memory 402 including many common features such as, for example, contacts 404, a calendar 406, a call log (or, call history) 408, and OS 410. In this case, the memory 402 may also store transplant app 412.

The user device 120 may also comprise one or more processors 416. In some implementations, the processor(s) 416 may be a central processing unit (CPU), a graphics processing unit (GPU), or both CPU and GPU, or any other sort of processing unit. The user device 120 may also include one or more of removable storage 418, non-removable storage 420, one or more transceiver(s) 422, output device(s) 424, and input device(s) 426.

In various implementations, the memory 402 may be volatile (such as random-access memory (RAM)), non-volatile (such as read only memory (ROM), flash memory, etc.), or some combination of the two. The memory 402 may include all, or part, of the functions 404, 406, 408, 412, and the OS 410 for the user device 120, among other things.

The memory 402 may also comprise contacts 404, which may include names, numbers, addresses, and other information about the user's business and personal acquaintances, among other things. In some examples, the memory 402 may also include a calendar 406, or other software, to enable the user to track appointments and calls, schedule meetings, and provide similar functions. In some examples, the memory 402 may also comprise the call log 408 of calls received, missed, and placed from the user device 120. As usual, the call log 408 may include timestamps for each call for use by the system 100. Of course, the memory 402 may also include other software such as, for example, e-mail, text messaging, social media, and utilities (e.g., calculators, clocks, compasses, etc.).

The memory 402 may also include the OS 410. Of course, the OS 410 varies depending on the manufacturer of the user device 120 and currently comprises, for example, iOS 12.1.4 for Apple products and Pie for Android products. The OS 410 contains the modules and software that supports a computer's basic functions, such as scheduling tasks, executing applications, and controlling peripherals.

As mentioned above, the user device 120 may also include the transplant app 412. The transplant app 412 may perform some, or all, of the functions discussed above with respect to the methods 200, 300A and 300B for interactions occurring between the user device 120 and the computing device 110. Thus, the transplant app 412 may receive a graph (e.g., the first graph), a message (e.g., the first message), and details about the organ. The transplant app 412 may also send a response (e.g., first response) and may also allow the user to enter certain characteristics (e.g., age, height, weight, blood type, medical history, family medical history, etc.) and send those characteristics to the computing device 110 and/or the external server 130.

The user device 120 may also include additional data storage devices (removable and/or non-removable) such as, for example, magnetic disks, optical disks, or tape. Such additional storage is illustrated in FIG. 4 by removable storage 418 and non-removable storage 420. The removable storage 418 and non-removable storage 420 may store some, or all, of the functions 404, 406, 408, 412, and the OS 410.

Non-transitory computer-readable media may include volatile and nonvolatile, removable and non-removable tangible, physical media implemented in technology for storage of information, such as computer readable instructions, data structures, program modules, or other data. The memory 402, removable storage 418, and non-removable storage 420 are all examples of non-transitory computer-readable media. Non-transitory computer-readable media include, but are not limited to, RAM, ROM, electronically erasable programmable ROM (EEPROM), flash memory or other memory technology, compact disc ROM (CD-ROM), digital versatile disks (DVD) or other optical storage, magnetic cassettes, magnetic tape, magnetic disk storage or other magnetic storage devices, or any other tangible, physical medium which may be used to store the desired information and which may be accessed by the user device 120. Any such non-transitory computer-readable media may be part of the user device 120 or may be a separate database, databank, remote server, or cloud-based server.

In some implementations, the transceiver(s) 422 include any sort of transceivers known in the art. In some examples, the transceiver(s) 422 may include wireless modem(s) to facilitate wireless connectivity with the other user devices, the Internet, and/or an intranet via a cellular connection.

In other examples, the transceiver(s) 422 may include wired communication components, such as a wired modem or Ethernet port, for communicating with the other user devices or the provider's Internet-based network. In this case, the transceiver(s) 422 may also enable the user device 120 to communicate with the computing device 110 and the external server 130, as described herein.

In some implementations, the output device(s) 424 include any sort of output devices known in the art, such as a display (e.g., a liquid crystal or thin-film transistor (TFT) display), a touchscreen display, speakers, a vibrating mechanism, or a tactile feedback mechanism. In some examples, the output device(s) 424 may play various sounds based on, for example, whether the user device 120 is connected to a network, the type of call being received (e.g., video calls vs. voice calls), the number of active calls, etc. In some examples, the output device(s) may play a sound when the message including the offer for the organ is received, when the graph is downloaded, etc. Output device(s) 424 may also include ports for one or more peripheral devices, such as headphones, peripheral speakers, or a peripheral display.

In various implementations, input device(s) 426 may include any sort of input devices known in the art. The input device(s) 426 may include, for example, a camera, a microphone, a keyboard/keypad, or a touch-sensitive display. A keyboard/keypad may be a standard push button alphanumeric, multi-key keyboard (such as a conventional QWERTY keyboard), virtual controls on a touchscreen, or one or more other types of keys or buttons, and may also include a joystick, wheel, and/or designated navigation buttons, or the like.

As shown in FIG. 5, the system 100 and methods 200, 300A and 300B may also be used in conjunction with the computing device 110. The computing device 110 may comprise, for example, a desktop or laptop computer, a server, bank of servers, or cloud-based server bank. Thus, while the computing device 110 is depicted as single standalone servers, other configurations or existing components could be used.

In various implementations, the memory 502 may be volatile (such as random-access memory (RAM)), non-volatile (such as read only memory (ROM), flash memory, etc.), or some combination of the two. The memory 502 may include all, or part, of the functions of a transplant app 508, among other things. The memory 502 may also include the OS 510. Of course, the OS 510 varies depending on the manufacturer of the computing device 110 and the type of component. Many servers, for example, run Linux or Windows Server. The OS 510 contains the modules and software that supports a computer's basic functions, such as scheduling tasks, executing applications, and controlling peripherals.

The computing device 110 may also comprise one or more processors 516, which may include a central processing unit (CPU), a graphics processing unit (GPU), or both CPU and GPU, or any other sort of processing unit. The transplant app 508 may provide communication between the computing device 110 and the user device 120 and/or the external server 130. Thus, the transplant app 508 may send a graph (e.g., the second graph), a message (e.g., the second message), and details about the organ to the user device 120. Also, the transplant app 508 may receive a response to the message from the user device 120. Further, the transplant app 508 may receive characteristics of the prospective organ recipient from the user device 120 associated with the prospective organ recipient and/or from the external server 130.

The computing device 110 may also include additional data storage devices (removable and/or non-removable) such as, for example, magnetic disks, optical disks, or tape. Such additional storage is illustrated in FIG. 5 by removable storage 518 and non-removable storage 520. The removable storage 518 and non-removable storage 520 may store some, or all, of the OS 510 and functions 508.

Non-transitory computer-readable media may include volatile and nonvolatile, removable and non-removable tangible, physical media implemented in technology for storage of information, such as computer readable instructions, data structures, program modules, or other data. The memory 502, removable storage 518, and non-removable storage 520 are all examples of non-transitory computer-readable media. Non-transitory computer-readable media include, but are not limited to, RAM, ROM, EEPROM, flash memory or other memory technology, CD-ROM, DVDs or other optical storage, magnetic cassettes, magnetic tape, magnetic disk storage or other magnetic storage devices, or any other tangible, physical medium which may be used to store the desired information, and which may be accessed by the computing device 110. Any such non-transitory computer-readable media may be part of the computing device 110 or may be a separate database, databank, remote server, or cloud-based server.

In some implementations, the transceiver(s) 522 include any sort of transceivers known in the art. In some examples, the transceiver(s) 522 may include wireless modem(s) to facilitate wireless connectivity with the user device 120, the Internet, and/or an intranet via a cellular connection. Further, the transceiver(s) 522 may include a radio transceiver that performs the function of transmitting and receiving radio frequency communications via an antenna (e.g., Wi-Fi or Bluetooth®). In other examples, the transceiver(s) 522 may include wired communication components, such as a wired modem or Ethernet port, for communicating with the other user devices or the provider's Internet-based network. The transceiver(s) 522, may receive the first and/or the second dataset from the user device 120 and/or the external server 130.

In some implementations, the output device(s) 524 include any sort of output devices known in the art, such as a display (e.g., a liquid crystal or thin-film transistor (TFT) display), a touchscreen display, speakers, a vibrating mechanism, or a tactile feedback mechanism. In some examples, the output devices may play various sounds based on, for example, whether the computing device 110 is connected to a network, the type of data being received (e.g., the first dataset vs. the second dataset), when the message is being transmitted, etc. Output device(s) 524 also include ports for one or more peripheral devices, such as headphones, peripheral speakers, or a peripheral display.

In various implementations, input device(s) 526 include any sort of input devices known in the art. For example, the input device(s) 526 may include a camera, a microphone, a keyboard/keypad, or a touch-sensitive display. A keyboard/keypad may be a standard push button alphanumeric, multi-key keyboard (such as a conventional QWERTY keyboard), virtual controls on a touchscreen, or one or more other types of keys or buttons, and may also include a joystick, wheel, and/or designated navigation buttons, or the like.

The specific configurations, machines, and the size and shape of various elements may be varied according to particular design specifications or constraints requiring a user device 120, computing device 110, external server 130, system 100, or method 200, 300A, 300B constructed according to the principles of this disclosure. Such changes are intended to be embraced within the scope of this disclosure. The presently disclosed examples, therefore, are considered in all respects to be illustrative and not restrictive. The scope of the disclosure is indicated by the appended claims, rather than the foregoing description, and all changes that come within the meaning and range of equivalents thereof are intended to be embraced therein.

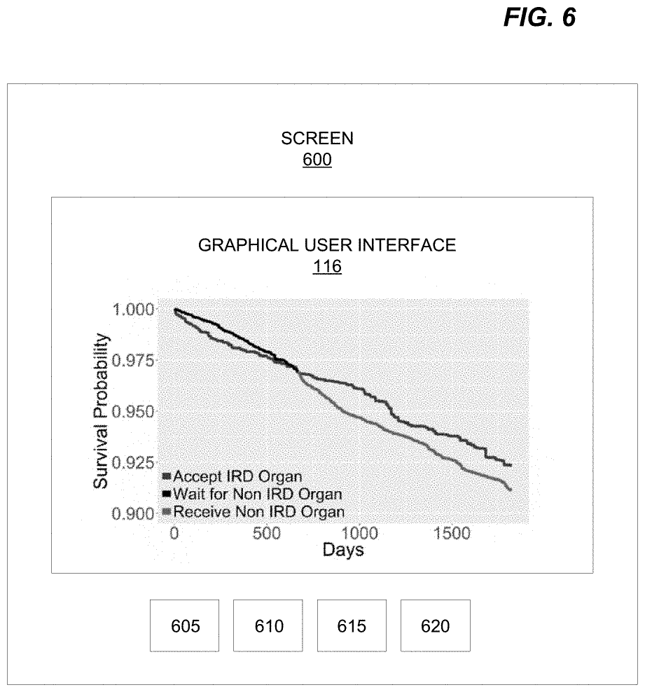

FIG. 6 depicts an example of a screen 600 that includes a graphical user interface 116 for use with the system 100, user devices 120A-n, and methods 200, 300A and 300B disclosed herein. The screen 600 may be included as part of, for example, the user device 120, for example, or the computing device 110. In addition to the GUI 116, the screen 600 may also include a plurality of buttons 605, 610, 615, and 620. The buttons may be visual representations of certain keys on a keyboard and/or may be specific functions, for example, button 605 may be an “acceptance button,” button 610 may be a “denial button,” button 615 may be an “insert button,” and button 620 may be a “select button.”

The GUI 116 may display the graph, which may include the first set of estimated survival rates and the second set of estimated survival rates, which the user may observe to determine whether to accept or deny the offer for the organ. The user may accept the offer by pressing button 605, or alternately the user may deny the offer by pressing button 610. The user may select, with button 620, a displayed field and then enter data (e.g., recent health status) by selecting the insert button 615.

Throughout the specification and the claims, the following terms take at least the meanings explicitly associated herein, unless the context clearly dictates otherwise. The term “or” is intended to mean an inclusive “or.” Further, the terms “a,” “an,” and “the” are intended to mean one or more unless specified otherwise or clear from the context to be directed to a singular form.

In this description, numerous specific details have been set forth. It is to be understood, however, that implementations of the disclosed technology can be practiced without these specific details. In other instances, well-known methods, structures and techniques have not been shown in detail in order not to obscure an understanding of this description. References to “one embodiment,” “an embodiment,” “some embodiments,” “example embodiment,” “various embodiments,” “one implementation,” “an implementation,” “example implementation,” “various implementations,” “some implementations,” etc., indicate that the implementation(s) of the disclosed technology so described can include a particular feature, structure, or characteristic, but not every implementation necessarily includes the particular feature, structure, or characteristic. Further, repeated use of the phrase “in one implementation” does not necessarily refer to the same implementation, although it can.

As used herein, unless otherwise specified the use of the ordinal adjectives “first,” “second,” “third,” etc., to describe a common object, merely indicate that different instances of like objects are being referred to, and are not intended to imply that the objects so described must be in a given sequence, either temporally, spatially, in ranking, or in any other manner.

While certain implementations of the disclosed technology have been described in connection with what is presently considered to be the most practical and various implementations, it is to be understood that the disclosed technology is not to be limited to the disclosed implementations, but on the contrary, is intended to cover various modifications and equivalent arrangements included within the scope of the appended claims. Although specific terms are employed herein, they are used in a generic and descriptive sense only and not for purposes of limitation.

This written description uses examples to disclose certain implementations of the disclosed technology, including the best mode, and also to enable any person skilled in the art to practice certain implementations of the disclosed technology, including making and using any devices or systems and performing any incorporated methods. The patentable scope of certain implementations of the disclosed technology is defined in the claims, and can include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1) A method for predicting survival rates of a prospective organ recipient, the method comprising:
receiving, by one or more processors of a computing device, a first dataset, the first dataset including characteristics of previous prospective organ transplant recipients, characteristics of previous organs received by the previous prospective organ transplant recipients, characteristics of previous persons in need of an organ transplant that did not receive the organ transplant, a first set of actual survival rates of the previous prospective organ transplant recipients, a second set of actual survival rates of the previous persons in need of an organ transplant that did not receive the organ transplant;
receiving, by the one or more processors, a second dataset, the second dataset including characteristics of the prospective organ recipient, characteristics of a first organ from a first organ donor available for an organ transplant into the prospective organ recipient, and an estimated wait time for a second organ from a second organ donor available for transplant into the prospective organ recipient to become available;
calculating, by the one or more processors executing a predictive algorithm, a first set of estimated survival rates of the prospective organ recipient over predetermined time periods based on at least a portion of the first dataset and at least a portion of the second dataset, the first set of estimated survival rates based on the organ recipient foregoing the organ transplant with the first organ;
calculating, by the one or more processors executing the predictive algorithm, a second set of estimated survival rates of the prospective organ recipient over the predetermined time periods based on at least a portion of the first dataset and at least a portion of the second dataset, the second set of estimated survival rates based on the organ recipient receiving the organ transplant with the first organ;
generating, by the one or more processors, a graph of the first set of estimated survival rates and the second set of estimated survival rates of the prospective organ recipient over the predetermined time periods; and
displaying, by a graphical user interface of the computing device, the graph.

2) The method of claim 1, wherein the characteristics of the previous prospective organ transplant recipients, the characteristics of previous organ donors received by the previous prospect organ transplant recipients, the characteristics of the prospective organ recipient, the characteristics of previous persons in need of an organ transplant that did not receive the organ transplant, and the characteristics of the first organ from the first organ donor available for an organ transplant include at least one of: age, sex, blood type, transplant region, height, weight, a medical condition, or an organ type, the organ type being a kidney, liver, lungs, or a heart.

3) The method claim 2, wherein the organ type further includes a status of the organ type, the status being infection-risk disease (IRD) or non-IRD.

4) The method of claim 1, wherein the first organ is a kidney, and wherein calculating the first set of estimated survival rates further comprises:
assigning, by the one or more processors, a respective weight to each of the characteristics of previous persons in need of an organ transplant that did not receive the organ transplant;
comparing, by the one or more processors, the characteristics of the prospective organ recipient to each of the characteristics of previous persons in need of an organ transplant that did not receive the organ transplant to determine at least partial congruence;
calculating, by the one or more processors, an average survival rate for the congruent previous persons that did not receive the organ transplant based on the second set of actual survival rates for each of the congruent previous persons; and
setting, by the one or more processors, the average survival rate as the first set of estimated survival rates.

5) The method of claim 1, wherein the first organ is a liver, heart, or lung, and wherein calculating the first set of estimated survival rates further comprises:
assigning, by the one or more processors, a respective weight to each of the characteristics of previous persons in need of an organ transplant that did not receive the organ transplant;
comparing, by the one or more processors, the characteristics of the prospective organ recipient to each of the characteristics of previous persons in need of an organ transplant that did not receive the organ transplant to determine at least partial congruence;
calculating, by the one or more processors, a transplant score for each of the prospective organ recipients by totaling the respective weights of congruent characteristics;
determining, by the one or more processors, an average set of survival rates for the previous persons in need of an organ transplant that did not receive the organ transplant;
calculating, by the one or more processors, new shifted survival rates by multiplying the average set of survival rates by the transplant score for each prospective organ recipient; and
setting, by one or more processors, the new shifted survival rates as the first set of estimated survival rates.

6) The method of claim 1, wherein the first organ is a kidney, and wherein calculating the second set of estimated survival rates further comprises:
assigning, by the one or more processors, a respective weight to each of the characteristics of previous organ transplant recipients;
comparing, by the one or more processors, the characteristics of the prospective organ recipient to each of the characteristics of previous organ transplant recipients to determine at least partial congruence;
calculating, by the one or more processors, an average survival rate for the congruent previous organ transplant recipients based on the actual survival rates for each of the congruent previous persons; and
setting, by the one or more processors, the average set of survival rates as the second set of estimated survival rates.

7) The method of claim 1, wherein the first organ is a liver, heart, or lung, and wherein calculating the second set of estimated survival rates further comprises:
assigning, by the one or more processors, a respective weight to each of the characteristics of previous organ transplant recipients;
comparing, by the one or more processors, the characteristics of the prospective organ recipient to each of the characteristics of previous organ transplant recipients to determine at least partial congruence;
calculating, by the one or more processors, a transplant score for each of the prospective organ recipients by totaling the respective weights of congruent characteristics;
determining, by the one or more processors, an average set of survival rates for the previous organ transplant recipients;
calculating, by the one or more processors, new shifted survival rates by multiplying the average set of survival rates by the transplant score for each prospective organ recipient; and
setting, by one or more processors, the new shifted survival rates as the second set of estimated survival rates.

8) The method of claim 1, further comprising:
sending, with a transceiver of the computing device, a message to a user device associated with the prospective organ transplant recipient, the message including at least one of: (i) an offer of the first organ, or (ii) instructions that cause a graphical user interface of the user device to display the graph; and
receiving, at the transceiver, a response from the user device associated with the prospective organ transplant recipient, the response including an acceptance or a denial.

9) The method of claim 1, further comprising:
sending, with a transceiver and to a server, the graph for retrieval by an application of a user device associated with the prospective organ transplant recipient.

10) A method for determining an organ recipient, the method comprising:
receiving, by one or more processors of a computing device, a first dataset, the first dataset including characteristics of previous prospective organ transplant recipients, characteristics of previous organs received by the previous prospective organ transplant recipients, characteristics of previous persons in need of an organ transplant that did not receive the organ transplant, a first set of actual survival rates of the previous prospective organ transplant recipients, a second set of actual survival rates of the previous persons in need of an organ transplant that did not receive the organ transplant;
receiving, by the one or more processors, a second dataset, the second dataset including characteristics of each of a plurality of prospective organ recipients and characteristics of an organ from an organ donor available for an organ transplant into at least one of the plurality of prospective organ recipients;
calculating, by the one or more processors executing a predictive algorithm, a first set of estimated survival rates for each of the plurality of prospective organ recipients over a predetermined period based at least on a portion of the first dataset and at least a portion of the second dataset, the first set of estimated survival rates based on each of the plurality of prospective organ recipients foregoing the organ transplant;
calculating, by the one or more processors executing the predictive algorithm, a second set of estimated survival rates for each of the plurality of prospective organ recipients over the predetermined period based at least on a portion of the first dataset and at least a portion of the second data, the second set of estimated survival rates based on each of the plurality of prospective organ recipients receiving the organ transplant;
identifying, by the one or more processors, an organ recipient from amongst the plurality of prospective organ recipients, the organ recipient having the highest second set of estimated survival rates over the predetermined period;
generating, by the one or more processors, a first graph of the first set of estimated survival rates of the organ recipient and the second set of estimated survival rates for the organ recipient over the predetermined period;
sending, with a transceiver of the computing device, the first graph and a first message to a first user device associated with the organ recipient, the first message including an offer of an organ; and
receiving, at the transceiver, a first response from the first user device, the first response including an acceptance or a denial of the offer of the organ.

11) The method of claim 10, further comprising:
sending, with the transceiver and to a server, the first graph for retrieval by an application of the first user device associated with the organ recipient.

12) The method of claim 10, wherein the characteristics of the previous prospective organ transplant recipients, the characteristics of previous organ donors received by the previous prospect organ transplant recipients, the characteristics of the prospective organ recipient, the characteristics of previous persons in need of an organ transplant that did not receive the organ transplant, and the characteristics of the organ from the organ donor available for an organ transplant include at least one of: age, sex, blood type, transplant region, height, weight, a medical condition, or an organ type, the organ type being a kidney, liver, lungs, or a heart.

13) The method claim 12, wherein the organ type further includes a status of the organ type, the status being infection-risk disease (IRD) or non-IRD.

14) The method of claim 10, wherein upon receiving a denial, the method further comprises:
determining, by the one or more processors, a next organ recipient from the plurality of prospective organ recipients, the next organ recipient having the second highest second set of estimated survival rates over the predetermined period.

15) The method of claim 14, further comprising:
sending, with the transceiver, a second message to a second user device associated with the next organ recipient, the second message including an offer of the organ;
receiving, at the transceiver, a second response from the second user device, the second response including an acceptance or a denial.

16) The method of claim 14, further comprising:
identifying, by the one or more processors, the first set of estimated survival rates for the next organ recipient from the first set of estimated survival rates; and
identifying, by the one or more processors, the second set of estimated survival rates for the next organ recipient from the second set of estimated survival rates.

17) The method of claim 16, further comprising:
generating, by the one or more processors, a second graph of the first set of estimated survival rates of the next organ recipient and the second set of estimated survival rates for the next organ recipient over the predetermined period;
sending, with the transceiver, the second graph and a second message to a second user device associated with the next organ recipient, the second message including an offer of the organ;
receiving, at the transceiver, a second response from the second user device, the second response including an acceptance or a denial of the offer of the organ.

18) The method of claim 17, further comprising:
sending, with the transceiver and to a server, the second graph for retrieval by an application of the second user device.

19) A system for predicting survival rates of a prospective organ recipient, the system comprising:
one or more processors;
a transceiver;
a graphical user interface operably connected to the one or more processors; and
a memory in communication with the one or more processors and the transceiver, and storing instructions that, when executed by the one or more processors, are configured to:
receive, a first dataset, the first dataset including characteristics of previous prospective organ transplant recipients, characteristics of previous organs received by the previous prospective organ transplant recipients, characteristics of previous persons in need of an organ transplant that did not receive the organ transplant, a first set of survival rates of the previous prospective organ transplant recipients, a second set of survival rates of the previous persons in need of an organ transplant that did not receive the organ transplant;
receiving, a second dataset, the second dataset including characteristics of the prospective organ recipient, characteristics of a first organ from a first organ donor available for an organ transplant into the prospective organ recipient, and an estimated wait time for a second organ from a second organ donor available for transplant into the prospective organ recipient to become available;
calculate, using a predictive algorithm, a first set of estimated survival rates of the prospective organ recipient over predetermined time periods based on at least a portion of the first dataset and at least a portion of the second dataset, the first set of estimated survival rates based on the organ recipient foregoing the organ transplant with the first organ;
calculate, using the predictive algorithm, a second set of estimated survival rates of the prospective organ recipient over the predetermined time periods based on at least a portion of the first dataset and at least a portion of the second dataset, the second set of estimated survival rates based on the organ recipient receiving the organ transplant with the first organ;
generate a graph of the first set of estimated survival rates and the second set of estimated survival rates of the prospective organ recipient over the predetermined time period;
send the graph to a server for retrieval by an application of a user device associated with the prospective organ transplant recipient; and
cause the graphical user interface to display the graph.

20) The system of claim 19, wherein the one or more processors is further configured to:
send the graph and a message to the user device, the message including an offer of an organ;
receive a response from the user device, the response including an acceptance or a denial.
